(19) United States
(12) Patent Application Publication
(10) Pub. No.: US 2012/0012522 A1
(43) Pub. Date: Jan. 19, 2012

(54) MAKING AND USING COMPOSITE MATERIAL CONTAINING NANOSPHERES AND DEVICES FOR WATER FILTRATION AND DEVICES CONTAING SUCH COMPOSITES

(76) Inventors: Sabyasachi Sarkar, Jamtara (IN); Abdul Allam, Raleigh, NC (US); Afreen Allam, Raleigh, NC (US); Iffat Allam, Raleigh, NC (US)

(21) Appl. No.: 13/181,855

(22) Filed: Jul. 13, 2011

Related U.S. Application Data
(60) Provisional application No. 61/365,031, filed on Jul. 16, 2010.

(51) Int. Cl. B01D 39/06; C01B 31/02; C07F 5/06; B32B 5/16; C07C 51/275; C07F 15/02; B82Y 30/00; B82Y 40/00

(52) U.S. Cl. 210/503; 562/400; 423/445 R; 556/138; 556/170; 428/402; 210/500.1; 977/896; 977/773; 977/902

(57) ABSTRACT

The present invention relates to the method of producing concentric carbon nanospheres from the pyrolytic combustion of a carbonaceous material such as plant material. The material can be carboxylated and then optionally metallated to produce nanospheres capable of filtering a liquid such as water.

This application claims priority of U.S. provisional application No. 61/365,031 filed on Jul. 16, 2010 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the production of carbon nanospheres and to use of the nanospheres in water filters and methods of filtering water using the composition in a water filter device.

2. Description of Related Art

Anthropogenic activities have caused severe changes in the natural state of resources in the environment. Essential resources like water and air became unfit for human consumption through pollution. Furthermore, uncontrolled tapping of aquifer for drinking water and its excessive misuse release dormant arsenic or fluoride ion into groundwater finally contaminating surface water and creating potentially serious environmental problems for humans and other living organisms trying to find clean drinking water. Further, pesticides and herbicides accumulate in soil and leach into ground water and water runoff. This is even further aggravated by uncontrolled damping of used electronic gadgets and batteries in soil. Furthermore, through leakage of water pipes, drinking water contamination with the open sewerage system becomes infected with feces containing pathogenic microbes. Such contaminations are a health risk and there is an urgent demand for a highly effective, reliable, and economical technique for the removal of toxic elements and pathogens from drinking water.

A number of methods have been developed to filter or otherwise remove pollutions and unwanted elements from drinking water. Included are methods of precipitation, ceramic or other filters, calcium or magnesium hydroxide, activated carbon, nano silver, chemical reaction, and the like. These materials are frequently used for incorporation as filter material in a filter device such as a housing containing filter material where water can be passed through for filtration purposes. The size of the particle filtered out is directly related to the porosity of the filtering material. Filtration of water is typically divided into nano porous, meso porous, micro porous, and the like.

While these methods and materials each have their benefits, they all tend to be limited in what they will filter, in some cases there have been negative health implications and/or high cost and use issues that do not make them the most ideal candidate for purifying drinking water.

### BRIEF SUMMARY OF THE INVENTION

The present invention provides novel filtration material that is cost effective and environmentally friendly, and filtration devices made from those materials. The materials are porous materials that can be used in water filtration devices and to filter other liquids. The porous nanospheres, the nanospheres with carboxyl groups and/or with metallated carboxy groups, can each together or separate be effective in not only filtering water for particulate matter, but in the case of the metallated nanospheres, binding and thus removing arsenic and fluoride and other materials from water passed through such filter material.

Accordingly, in one embodiment the present invention relates to a material for use as a filtering media comprising concentric carbon nanospheres, the outermost sphere in the size range of about 25 nm to about 50 nm which have been produced by the pyrolysis of a carbonaceous material.

In another embodiment, the present invention relates to a method for the production of a composite for use in filtering water comprising producing and isolating concentric carbon nanospheres from the pyrolysis of a carbonaceous material with a size range of the outermost sphere of 25 nm to 50 nm.

### OBJECTS OF THE INVENTION

An object of the present invention is to provide low cost bulk synthesis of concentric carbon nano spheres (CNSs) from readily available economical carbonaceous sources that enables easy industrial production of such CNSs in high yield and high mass-productivity especially for use in filtration, such as water filtration.

Another object of the present invention is to carboxylate these CNSs by oxidative treatment.

A further object of the present invention is to incorporate metallation using metal ions such as soluble ferric ion and aluminum ion to get a metallated carboxylate derivative of CNSs.

Further, the derivatized (extended with ionic fillers like quaternary ammonium salts or lithium chloride) or non-derivatized CNS a 2-40% is mixed with paper or wood pulp and compressed under heat to shape as a thin sheet of CNS paper that can be used for EMI protection.

It is a further object to create a water filtration material that traps or binds arsenic and/or fluoride or other materials present in water being filtered.

### BRIEF DESCRIPTION OF THE DRAWINGS

FIG. 1 depicts water soluble carbon nanospheres (CNSs) showing D and G band with mixing of overtones around 2700 cm −1 in Raman Spectrum.

FIG. 2 is an EDX spectra of CNSs showing the presence of only carbon and oxygen.

FIG. 3 is a SEM Image of pure Carbon Nanospheres.

FIG. 4 is a TEM image of Carbon Nanospheres.

FIG. 5 is a HRTEM image of CNSs showing sizes of the nanospheres with onion type multilayered concentric nanospheres.

FIG. 6 is a SEM image of the carboxylated CNSs showing size and shape.

FIG. 7 is a SEM image of carboxy metallated CNSs showing sizes of the nanospheres.

FIG. 8 is a pictorial representation of the metallation of carboxylated carbon nanosphere.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### Definitions

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The term “about” means±10% unless noted otherwise.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the terms and phrases “filtration”, “filtration of water”, and “water filter” refer to material that can be utilized to filter a liquid such as water. The material can be placed/positioned in a filtration device such as a water filter and liquid passed through the filtration material in the normal manner for water filtration. The material can be shaped, placed, containerized, or the like as needed to filter water under gravity pressure or other forced pressure. In other embodiments, liquids other than water that are compatible with carbon filtration material can also be filtered.

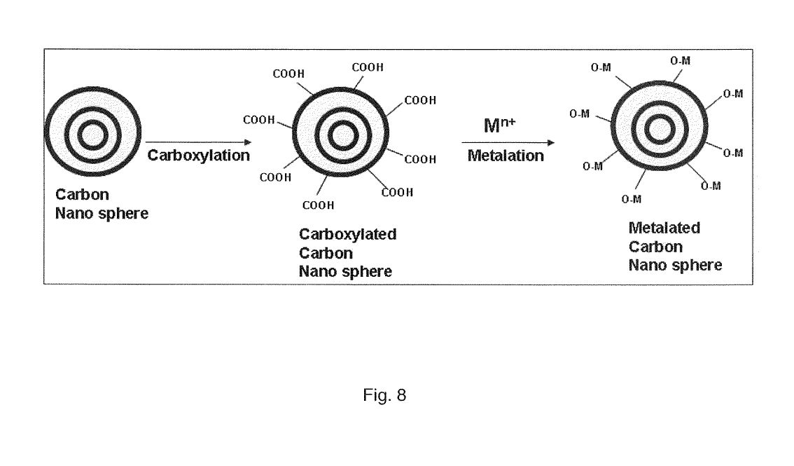

As used herein the term “nanospheres” refers to concentric spherical carbon structures which outermost spheres are in the size range of about 25 nm to about 50 nm that are isolated from the pyrolytic combustion of a carbonaceous material. The pyrolytic combustion produces concentric spheres that are two or more spheres inside another (see FIG. 8). When collected in a container in quantity (such as being placed in a filter), the nanospheres fill a given space like marbles fill a container, creating the minimal amount of space possible and aligning themselves due to their spherical shape. The porosity of the material is derived by spaces in-between the spheres since there is a consistant size of that space when packing spherical objects. The space between the spheres can be adjusted, that is made smaller, thus filtering smaller by placing addition groups on the surface of the spheres, thus making them fuzzy and thus taking up space in the space between the spheres. The present invention includes carboxylation of the spheres wherein carboxylation is obtained by oxidation of the spheres, there being more carboxylation of the spheres the longer the spheres remain in contact with the oxidizing agent (such as nitric acid). The carboxylation, once it reaches a certain point, makes the spheres water soluble, therefore, in the case where they are to be utilized for water filtration, the carboxylation should be kept below an amount that makes them soluble in the water being filtered. That amount can easily be determined by varying the oxidation time and testing a batch. Where a fluid other than water is being filtered, the amount of carboxylation can be less important.

The carboxylate groups can also be metallated. Metallation will cause the spheres to be non-water soluble so in that instance as much carboxylation as desired can be done if it is going to be metalized. One can combine these additions to the spheres having all carboxylation or metallization on a sphere, a combination of the two on a sphere, or a combination of spheres that are each all carboxylated or metallated. The non substituted spheres and these substituted spheres can be mixed together in any combination desired depending on the porosity desired. The more substitutions, the smaller the spaces and the smaller particle that is filtered out by the material.

In general, the material is loosely packed and can be placed in a cartridge or other container to hold it in place during the filtration process. In other embodiments, a binder can be utilized to make the spheres sticky enough to hold together as a composite. For example a silicic acid solution could coat the spheres without decreasing significantly the porosity of the material. Other binders could be determined by one skilled in the art.

As used herein the phrase “not carboxylated sufficiently to cause the carbon nano spheres to be water soluble” refers to only oxidizing the spheres enough so that the resulting spheres are still not soluble in water to a significant degree.

As used herein the terms “metal ion”, “metallate”, “metallation”, and “metallated” refer to treating the carboxylate groups place on nanospheres to place a metal ion on the group in place of the carboxylic group, i.e. replacing —COON with —COM wherein M is the metal ion. Metal ions can be selected from metals in the transition metals and poor metal groups from the periodic table. For example, iron, silver, and aluminum could be selected. The ions can be delivered in the form of the ion or a salt which dissociates to deliver the metal ion. It is known that certain ions can bind compounds and the selection of the metal ion can be done to selectively bind certain compounds dissolved in the liquid such as water in addition to filtering the media. For example, in some cases silver is known to attach to bacteria binding it, and in other cases actually killing it. Iron and aluminum can bind arsenic and fluoride respectively and can similarly be utilized. One skilled in the art can determine which metal to select based on binding capacity, cost, ease of use, and the like.

As used herein a “carbonaceous material” refers to material which when pyrolyzed, can form carbonaceous nanospheres (in the about 25 nm to about 50 nm size). In one embodiment the carbonaceous material is an organic material that is a material that was living such as animal or plant material. For example, the material could be a cellulosic material, wood, coconut shell, wood wool, wood dust, or the like. Other materials can also be utilized which can be pyrolyzed in sufficient quantity to make enough spheres to make a filter.

As used herein “oxidizing agent” refers to a composition that can oxidize the surface of a carbon spheres to produce carboxylic acid groups on the surface, for example concentrated or diluted nitric acid though there is damage to the spheres the more concentrated the acid is.

### Preparation of Nanospheres

Concentric Carbon nanospheres (CNS) are prepared by the pyrolysis of carbonaceous material under insufficient air. The carbonaceous material can be any material capable of pyrolysis (600 to 850 degrees C.) that produces nanospheres but in one embodiment is a natural material such as a biologic animal or plant material. Examples include but are not limited to coconut shell, wood wool, and wood dust. It can also include cellulose materials and wood.

In one embodiment, fire/heat is applied to the carbonaceous material in a confined chamber under the flow of blowing air where the carbonaceous matter is allowed to glow. An outlet on the top of the chamber continuously ejects the distillation byproducts such as ligneous acid under the flow of air from the bottom of the chamber maintained at a temperature around 600-850 degrees centigrade. The process takes several hours and is dependent on the quantity of the feed stock. When the oily byproduct ceases to come out through the top outlet, water is slowly introduced by sprinkling and the steam produced is dispersed or removed along with the residual byproduct. The residual mass is also cooled down. The mass is leached with alcohol to remove traces of soluble organic byproduct especially products like ligneous acid which can be trapped in the pours (spaces between the spheres) and air dried. It can also be washed with 10 percent sodium hydroxide solution for that purpose. The dried mass is pulverized to get the desired size of pieces containing multiple carbon nano spheres (macro sized in the millimeter range) by using appropriate mesh size while the individual spheres are together as shown in the SEM photographs. The CNS is now ready for further derivitization processes like carboxylation followed by metallation or can be utilized as is as a filtration material.

The collected black material from pyrolysis of the carbonaceous material which comprises amorphous carbon and carbon nanospheres is purified by washing. In one embodiment, ligneous acid and other impurities are removed with a washing of a solution of 10% sodium hydroxide. This can then subjected to a first derivitization by treating it with an oxidizing agent such as nitric acid to introduce multiple carboxylic acid groups on the surfaces of CNS. The longer the spheres are oxidized the greater the carboxylization. The carboxylated derivatized CNS are treated under sonication in water and are subjected to a second derivitization by adding metal ions such as ferric ion and aluminum ion and by adjusting the pH to metallate at least a portion the carboxylic acid groups. The formation can occur with the addition of ammonia followed by drying the mixture to a dried residue. Appropriate mixing and drying is done at each step. In most cases, what remains is a combination of spheres that are carboxylated, some that are metallated or a combination of the two. Non-deritivized spheres may remain as well. As noted, the filter material can contain any combination of the three types of nanospheres either because they all exist during producing them or because they are made separately and combined. The concentration of metal to CNS can vary from about 0.2 to about 10% weight/weight. The resultant mass is washed with water to remove free metal ions and other counter ions and heated around 100 degree centigrade to yield the metal impregnated form of CNS. See FIG. 8 for example. All these forms are activated carbon with assorted porosity.

### Creating a Filter

The material of the present invention can then be placed in a container or otherwise contained or formed as necessary for the desired filtration purpose or filter or filter holder. The shape can be as needed, for example to be placed in the flow of water to be filtered either by gravity or pressure filtration. For example, it can be contained in a standard filter holder or the like or custom holders for inline use can be created and such are unlimited in variety and shape. For example, where under gravity pressure for example thinner filters can be utilized up to 8 inches depending on how much must be filtered out. In the case of high pressure filtration virtually any thickness can be utilized as long as the pressure is not so great as to damage the filter material of the present invention. When CNS are prepared from low cost material, it can be utilized to develop eco-friendly filters that traps or binds soluble compounds of toxic metal ions and anions, pathogens and virus, and industrial organic molecules like pesticides and aromatic hydrocarbons besides bad color, odor in a unique integrated manner. The filter material of the present invention is also capable of reducing the salinity of water. The advantage of this invention over the comparable inventions lies on the following: The proposed system can remove inorganic, organic and bio related toxic materials commonly available in water to allow the water for drinking purpose. This requires no energy input as normally needed in systems that use ozone, ultraviolet radiation, chlorination, or reverse osmosis to remove pathogens and virus. The composition traps these pathogens in the pores and surface of CNS without need for any energy input.

The filter material which has bound materials such as arsenic and fluoride and bacteria to it can be chemically treated to regenerate the material. However, because of the low cost involved in the manufacture of the material, it can be readily disposed of or used as crude carbonaceous fuel if it no longer filters sufficiently for filtration utilization.

### EXAMPLES

### Example 1

CNS isolated from the pyrolytic combustion of coconut shells (by the method above) was treated with 50% nitric acid and 50% water mixture to introduce carboxylic acid groups due to oxidation which was evident by viewing a lot of brown fumes generated in the process showing reduction of the nitric acid. After few hours of standing from 1 to 8 hours (giving more or less carboxylation), varied density (number per unit of CNS) of carboxylation took place. The nitric acid is washed away with plenty of water till the mass is nitrate free and the black slurry is dried in air. This first time derivatized CNS (derivatized CNS1) in water under sonication was mixed with an aqueous solution of ferric salt and/or aluminum salt of varied proportion, followed by addition of ammonia to obtain a precipitate (in the pH range 7-8) to rid the mixture of the non metallic part of the salt. This was treated with plenty of water to remove the water soluble ions and dried in air to obtain a dried residue as second time derivatized CNS). Appropriate mixing of the nanospheres with different derivations is done under stirring. The resultant lump is crushed and heated to 100 degree centigrade to set silicate or cement to obtain the carbon nano composite.

### Example 2

1 mm sized globular alumina was treated with 10% aqueous solution of ferrous sulfate and allowed to soak for couple of hours. The slurry was made ammoniacal by adding 5% aqueous solution of ammonia whereby greenish black ferrous oxide precipitated out and adhered to the alumina surface. This material was then air dried whereby green ferrous oxide gets oxidized to brown ferric oxide. The iron oxide impregnated alumina was then heated in a muffle furnace at about 380 degree Centigrade to drive off or remove volatile ammonium sulfate and water and activate the material (iron oxide impregnated alumina) which can now be used for removal of Fluoride ions during filtration of water.

### Example 3

A protocol with reasonable rate of water flow is described here. 50 gm of the activated alumina impregnated with iron oxide was used to test its capacity to bind fluoride. Water containing 4 ppm (parts per million) level of fluoride ion was filtered at the rate of 5 liter of water per hour for the first stage of 100 liters of water. This was followed by 2 ppm level of fluoride for another 350 liters of water. The result was that 1.1 gm of fluoride was removed in this manner by using 50 gm of activated alumina impregnated with iron oxide filter bed. The flow rate is adjusted by using normal force of gravity without any external energy input source.

### Example 4

100 gms of the activated alumina impregnated with iron oxide filter bed, 53 liters of water containing 4 ppm fluoride (4 ppm means 12 mgs of potassium fluoride per liter of water) is used at initial stage of filtration. In the next stage 113 liters of water containing 2 ppm of fluoride (6 mgs of potassium fluoride per liter of water) in water is passed through the filter bed. It is shown that removal of fluoride is 2.2 gm measured as potassium fluoride.

### Example 5

Similarly using 50 gm of the modified ignited alumina impregnated with iron oxide as filter bed, water containing 5 ppm level of arsenic was passed through this bed at a flow rate of 5 liter of water per hour, measured grams of arsenic are removed from a passage of a quantity of water.

### EXPLANATIONS OF THE DRAWING AND FIGURES

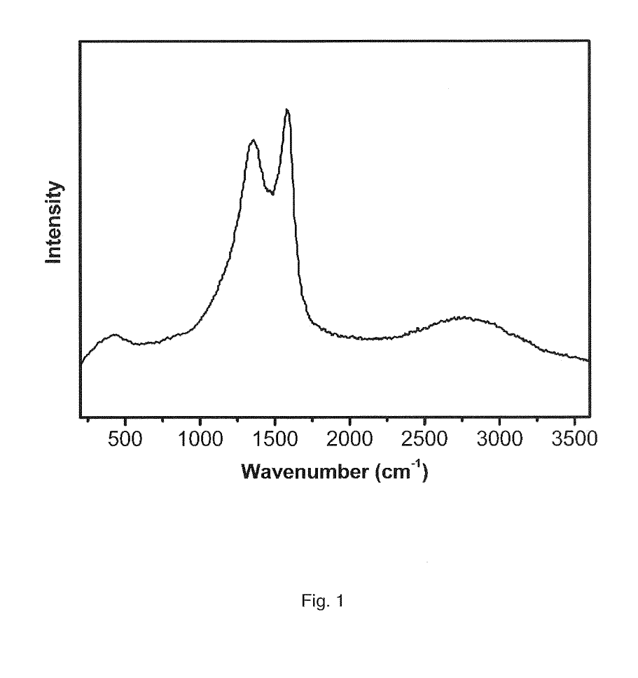

FIG. 1 is a graph of carboxylated concentric carbon nanospheres of the present invention showing D and G band with mixing of overtones around 2700 cm −1 in Raman spectrum.

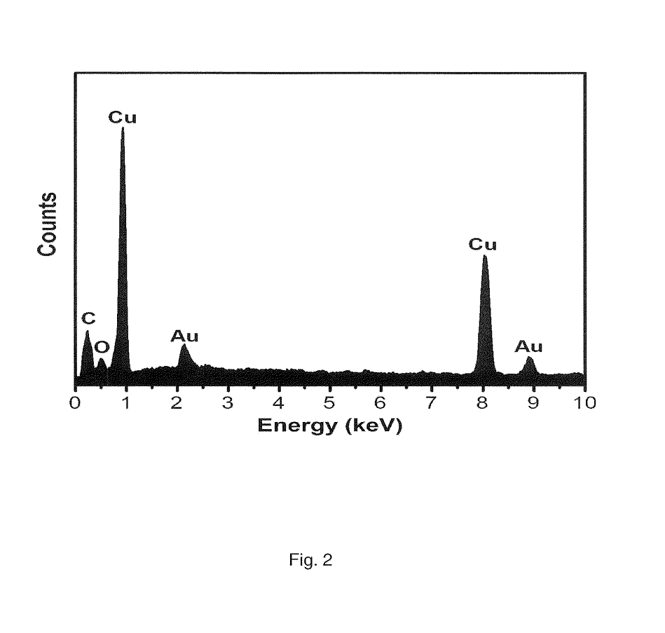

FIG. 2 is a chart of EDX of Carboxylated concentric carbon nanospheres of the present invention which shows the presence of oxygen and carbon.

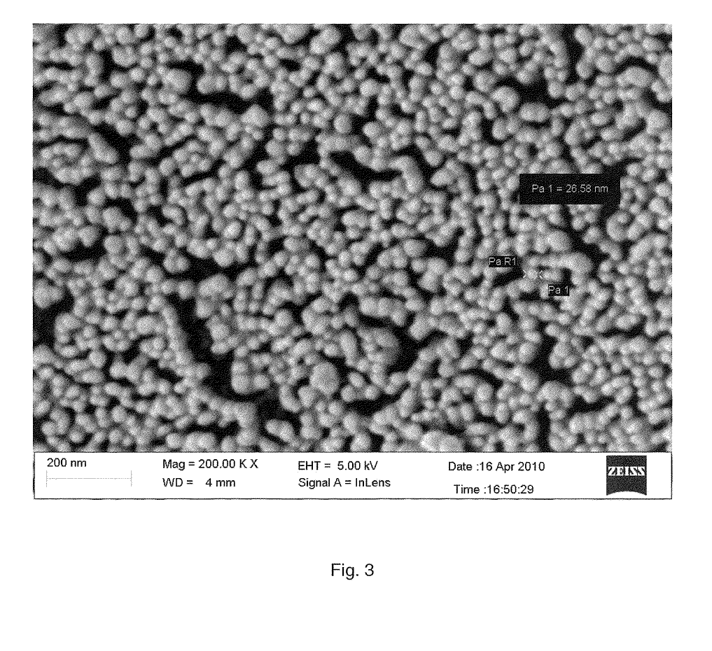

FIG. 3 is a SEM image of purified concentric carbon nanospheres of the present invention.

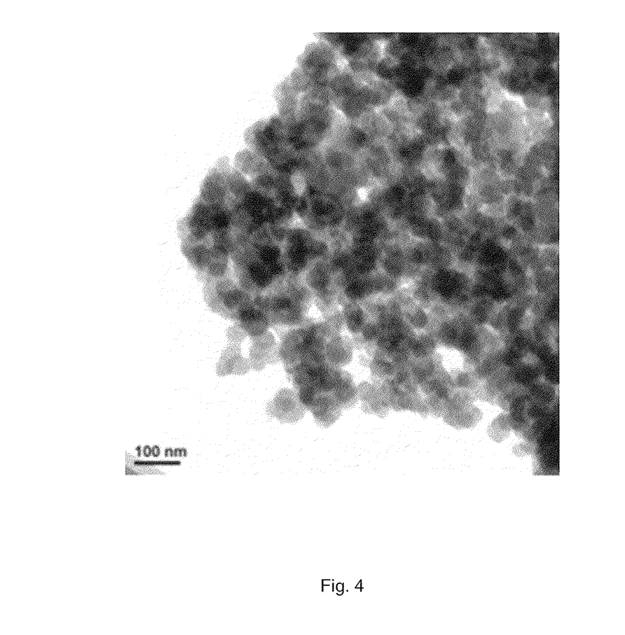

FIG. 4 is a TEM image of the purified nanospheres depicted in FIG. 3.

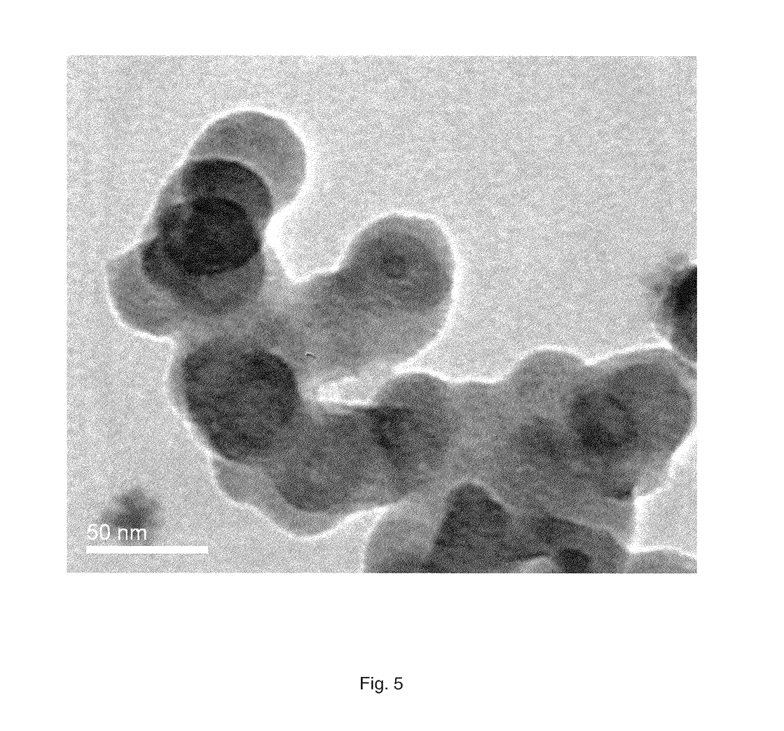

FIG. 5 shows a HRTEM image of the same purified concentric carbon nanospheres which shows their size is in the about 25 nm to about 50 nm range.

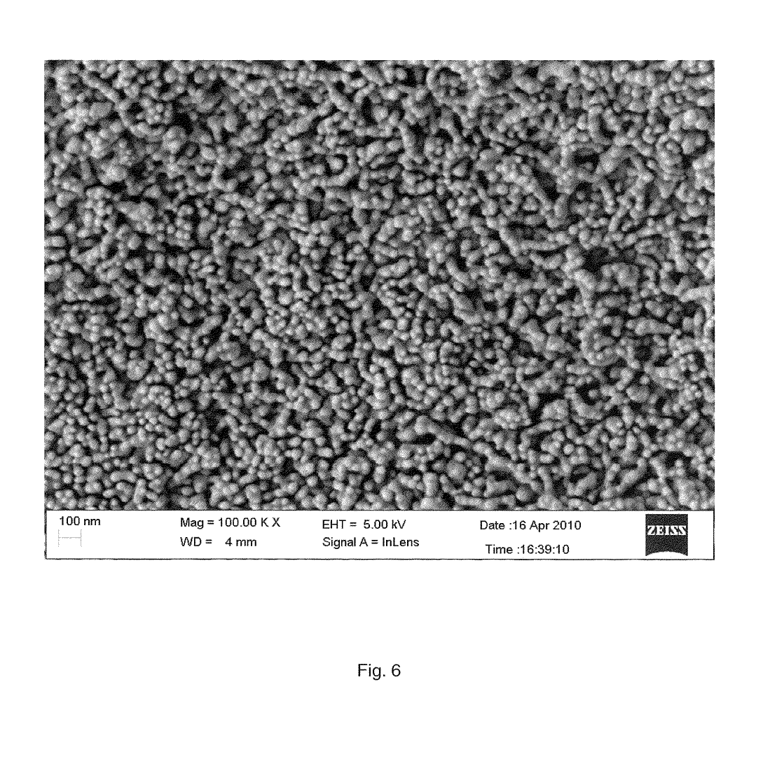

FIG. 6 shows a SEM image of the present invention carboxylated carbon nanosphere.

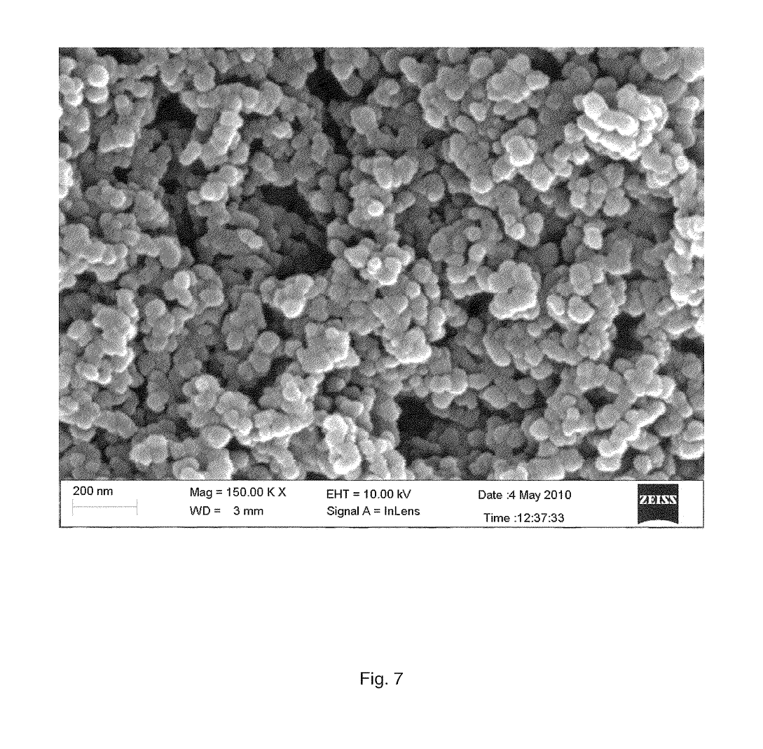

FIG. 7 shows a SEM image of metallated concentric carbon nanospheres of the present invention.

FIG. 8 is a graphic representation of the three types of concentric carbon nanospheres in cross section depicting the concentric spheres. In each sphere carbon nanospheres are carboxylated to produce carboxylated carbon nanospheres and then metallated to produce metallated carbon nanospheres.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A material for use as a filtering media comprising concentric carbon nanospheres the outermost sphere in the size range of about 25 nm to about 50 nm which have been produced by the pyrolysis of a carbonaceous material.

2. A material according to claim 1 wherein at least a portion of the carbon nanospheres have been at least partially carboxylated.

3. A material according to claim 1 wherein the carbon nanospheres are carboxylated and then a portion of the carboxylate groups metallated.

4. A material according to claim 1, 2 or 3 wherein the material comprises two or more of non-substituted nanospheres, carboxylated nanospheres, and carboxy metallated nanospheres wherein carboxylation and carboxy metallation can be on separate spheres or on the same spheres.

5. The composite according to claim 3 wherein the metallate is selected from the group comprising iron and aluminum each metal on each nanosphere being the same or different.

6. A water filter wherein the material according to claim 1, 2 or 3 is the filtering material in the filter.

7. A method for the production of a composite for use in filtering water comprising producing and isolating concentric carbon nanospheres from the pyrolysis of a carbonaceous material with a size range of the outermost sphere of 25 nm to 50 nm.

8. The method according to claim 1 wherein at least a portion of the nanospheres have at least partially been carboxylated.

9. The method according to claim 1 wherein at least a portion of the carboxylate groups are metallated.

10. The method according to claim 7 wherein the carbonaceous material is an organic carbon containing material.

11. The method according to claim 10 wherein the organic material is selected from the group comprising coconut shell, wood wool and wood dust.

12. The method according to claim 9 wherein the carboxylic acid groups are metallated with a soluble metallic ion from a metallic salt.

13. The method according to claim 12 wherein the metallic ion is one or more ion selected from the group comprising ferric ions and aluminum ions.

14. The method according to claim 7 wherein the composite is formed into a shape for use in a water filter.

15. The method according to claim 7 wherein the nanosheres are washed with a 10% solution of sodium hydroxide sufficient to remove impurities in the spaces between nanospheres.

16. The method according to claim 13 wherein iron oxide impregnated with alumina is used to metallate and is activated by heating at about 380 degrees C. sufficient to remove volatile ammonium sulfate and water and activate the material such that it binds fluoride ions.
